(19) United States
(12) Patent Application Publication
(10) Pub. No.: US 2024/0312079 A1
(43) Pub. Date: Sep. 19, 2024

(54) CONTENT EVALUATION DEVICE, COMPUTER-READABLE MEDIUM, METHOD, AND SYSTEM FOR EVALUATING CONTENT

(71) Applicant: Wacom Co., Ltd., Saitama (JP)

(72) Inventors: Ipei HUNG, Saitama (JP); Peter BACHER, Portland, OR (US); Jin-Fu KO, Saitama (JP)

(21) Appl. No.: 18/671,800

(22) Filed: May 22, 2024

Related U.S. Application Data
(63) Continuation of application No. PCT/JP2022/045095, filed on Dec. 7, 2022.

(30) Foreign Application Priority Data
Jan. 7, 2022 (JP) 2022-001486

(51) Int. Cl. G06T 11/20; A61B 5/16

(52) U.S. Cl. CPC G06T 11/203; A61B 5/165

(57) ABSTRACT

Provided is a content evaluation device including a processor and a memory storing a program that, when executed by the processor, causes the content evaluation device to: acquire at least one of content data indicating content composed of multiple content elements or related data relating to creation of the content, calculate a time-series feature indicating a time change of a feature relating to a creation process of the content from the content data or the related data, and evaluate the content by using the time-series feature.

### BACKGROUND

### Technical Field

The present disclosure relates to a content evaluation device, a computer-readable medium, a method, and a system for evaluating content.

### Description of the Related Art

Conventionally, a technique for allowing multiple users to share digital content (hereinafter, simply referred to also as “content”) that is an intangible object by using a computer system has been known (for example, refer to Japanese Patent No. 6734502).

Recently, along with the progress of artificial intelligence techniques, various machine learning models (as one example, generative adversarial networks; GANs) for generating content have been proposed. For example, it is anticipated that it becomes difficult to determine the authenticity of content merely by simply comparing the contents of drawing of a finished product if it becomes possible to generate an imitation of content automatically and elaborately by use of this kind of machine learning model.

### BRIEF SUMMARY

The present disclosure is made in view of such a problem and has a desire to provide a content evaluation device, a computer-readable medium, a method, and a system that can evaluate content more elaborately, compared with the case of executing evaluation merely by use of the contents of drawing of a finished product.

A content evaluation device in a first aspect of the present disclosure includes a processor and a memory storing a program that, when executed by the processor, causes the content evaluation device to: acquire at least one of content data indicating content composed of multiple content elements or related data relating to creation of the content, calculate a time-series feature indicating a time change of a feature relating to a creation process of the content from the content data or the related data, and evaluate the content by using the time-series feature calculated by the feature calculating section.

A content evaluation computer-readable medium in a second aspect of the present disclosure stores a content evaluation program that, when executed by one or more computers, causes the one or more computers to: acquire at least one of content data indicating content composed of multiple content elements or related data relating to creation of the content, calculate a time-series feature indicating a time change of a feature relating to a creation process of the content from the content data or the related data, and calculate the content by using the time-series feature.

A content evaluation method in a third aspect of the present disclosure includes, by one or multiple computers, acquiring at least one of content data indicating content composed of multiple content elements or related data relating to creation of the content, calculating a time-series feature indicating a time change of a feature relating to a creation process of the content from the content data or the related data, and evaluating the content by use of the time-series feature.

A content evaluation system in a fourth aspect of the present disclosure includes a user device that, in operation, generates content data indicating content composed of multiple content elements, and a server device that, in operation, communicates with the user device. The server device includes a processor; and a memory storing a program that, when executed by the processor, causes the server device to: acquire at least one of the content data or related data relating to creation of the content from the user device, calculate a time-series feature indicating a time change of a feature relating to a creation process of the content from the content data or the related data, and a content evaluating section that evaluates the content by using the time-series feature calculated by the feature calculating section.

According to the present disclosure, the content can be evaluated more elaborately, compared with the case of executing evaluation merely by use of the contents of drawing of a finished product.

### BRIEF DESCRIPTION OF THE SEVERAL VIEWS OF THE DRAWINGS

FIG. 1 is an overall configuration diagram of a content evaluation system in one embodiment of the present disclosure;

FIG. 2 is a block diagram illustrating one example of a configuration of a server device in FIG. 1;

FIG. 3 is a flowchart illustrating a first operation of the server device;

FIG. 4 is a diagram illustrating one example of content made by use of a user device in FIG. 1;

FIG. 5 is a diagram illustrating one example of a data structure that content data in FIG. 1 has;

FIG. 6 is a diagram illustrating one example of time change of a feature;

FIG. 7 is a diagram illustrating time change of the feature in the case in which a blank time is included in the creation period of content;

FIG. 8 is a diagram illustrating time change of the feature in the case in which the blank time has been cut down;

FIG. 9 is a diagram illustrating one example of a time-series feature calculated by a feature calculating section;

FIG. 10 is a diagram illustrating a correspondence relation between evaluation items and the feature; and

FIG. 11 is a flowchart illustrating second operation of the server device.

### DETAILED DESCRIPTION

An embodiment of the present disclosure will be described below with reference to the accompanying drawings. To facilitate understanding of the description, the same constituent element is given the same numeral as much as possible in the respective drawings, and overlapping description is omitted.

### Configuration of Content Evaluation System 10

### Overall Configuration

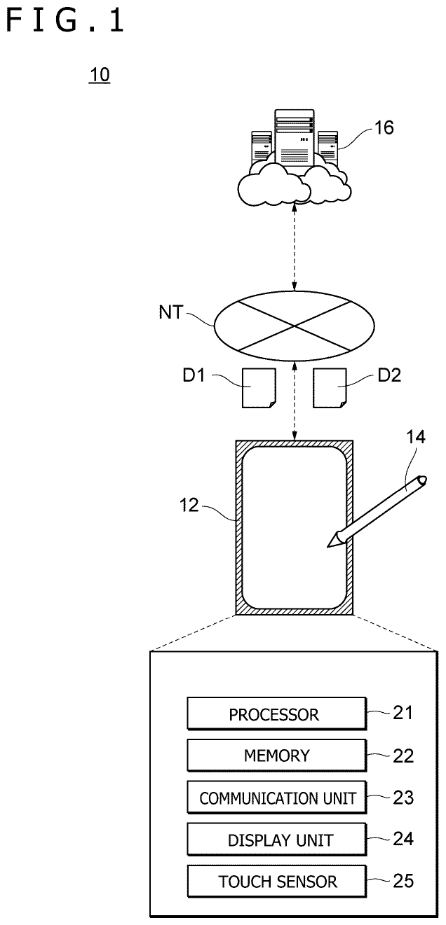

FIG. 1 is an overall configuration diagram of a content evaluation system 10 in one embodiment of the present disclosure. The content evaluation system 10 is made in order to provide a “content evaluation service” for evaluating computerized content (what is generally called digital content). Specifically, this content evaluation system 10 includes one or multiple user devices 12, one or multiple electronic pens 14, and a server device 16 (corresponding to a “content evaluation device”).

The user device 12 is a computer owned by a user (for example, a creator of content) who uses the content evaluation service and is configured from a tablet, a smartphone, a personal computer, or the like, for example. The user device 12 is configured to be capable of generating content data D1 and related data D2 to be both described later and supplying various kinds of data generated by the user device 12 to the server device 16 through a network NT. Specifically, this user device 12 includes a processor 21, a memory 22, a communication unit 23, a display unit 24, and a touch sensor 25.

The processor 21 is configured by a computation processing device including a central processing unit (CPU), a graphics processing unit (GPU), and a micro-processing unit (MPU). The processor 21 executes generation processing to generate digital ink that describes content, rendering processing to cause display of content indicated by digital ink, and so forth by reading out a program and data stored in the memory 22.

The memory 22 stores the programs and data necessary for the processor 21 to control the respective constituent elements. The memory 22 is configured from a non-transitory computer-readable storage medium. Here, the computer-readable storage medium is configured from [1] a storage device such as a hard disk drive (HDD) or a solid state drive (SSD) incorporated in a computer system, [2] a portable medium such as a magneto-optical disc, a read only memory (ROM), a compact disk (CD)-ROM, or a flash memory, or the like.

The communication unit 23 has a communication function to execute wired communication or wireless communication with an external device. This allows the user device 12 to, for example, exchange various kinds of data including the content data D1 or the related data D2 with the server device 16.

The display unit 24 can visibly display content including an image or video and is configured from a liquid crystal panel, an organic electro-luminescence (EL) panel, or electronic paper, for example. By allowing the display unit 24 to have flexibility, the user can execute various kinds of writing operation with a touch surface of the user device 12 remaining in a curved or bent state.

The touch sensor 25 is a sensor of a capacitive system obtained by disposing multiple sensor electrodes in a planner manner. For example, this touch sensor 25 includes multiple X line electrodes for detecting the position in an X-axis of a sensor coordinate system and multiple Y line electrodes for detecting the position in a Y-axis. The touch sensor 25 may be a sensor of the self-capacitance system in which block-shaped electrodes are disposed in a two-dimensional lattice manner instead of the above-described sensor of the mutual capacitance system.

The electronic pen 14 is a pen-type pointing device and is configured to be capable of unidirectionally or bidirectionally communicating with the user device 12. For example, this electronic pen 14 is a stylus of the active capacitance type (AES) system or the electromagnetic resonance (EMR) system. The user can write pictures, characters, and so forth to the user device 12 by gripping the electronic pen 14 and moving the electronic pen 14 while pressing the pen tip against the touch surface that the user device 12 has.

The server device 16 is a computer that executes comprehensive control relating to evaluation of content and may be either a cloud type or an on-premise type. Here, the server device 16 is illustrated as a single computer. However, the server device 16 may be a computer group that constructs a distributed system, instead of this.

### Block Diagram of Server Device 16

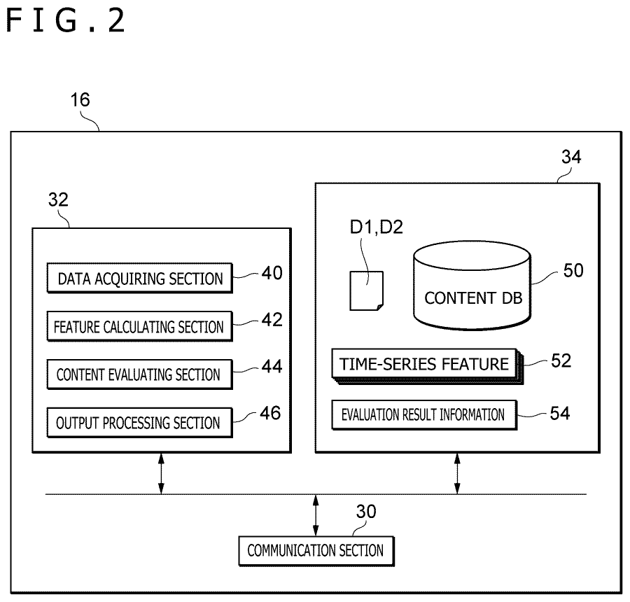

FIG. 2 is a block diagram illustrating one example of a configuration of the server device 16 in FIG. 1. Specifically, the server device 16 includes a communication section 30, a control section 32, and a storing section 34.

The communication section 30 is an interface that transmits and receives an electrical signal to and from an external device. This allows the server device 16 to acquire at least one of the content data D1 and the related data D2 from the user device 12 and provide evaluation result information 54 generated by the server device 16 to the user device 12.

The control section 32 is configured by a processor including a CPU and a GPU. The control section 32 functions as a data acquiring section 40, a feature calculating section 42, a content evaluating section 44, and an output processing section 46 by reading out a program and data stored in the storing section 34 and executing the program.

The data acquiring section 40 acquires various kinds of data (for example, content data D1, related data D2, and so forth) relating to content that is an evaluation target. The data acquiring section 40 may acquire the various kinds of data from an external device through communication or acquire the various kinds of data through reading out them from the storing section 34.

The feature calculating section 42 calculates a time-series feature 52 indicating a time change of a feature relating to the creation process of the content from the content data D1 or the related data D2 acquired by the data acquiring section 40. In this calculation, [1] “removal processing” to remove data unnecessary for the calculation of the time-series feature 52, [2] “association processing” to associate each feature with the time, [3] “cutting-down processing” to cut down a blank time according to need, [4] “normalization processing” to execute normalization of the feature and the time, or the like is executed.

The content evaluating section 44 executes evaluation processing to evaluate content by using the time-series feature 52 calculated by the feature calculating section 42. For example, the content evaluating section 44 evaluates [1] the style of content, [2] creator's habit, [3] the psychological state of the creator, or [4] the state of the external environment. Here, the “style” means individuality or thought of the creator that appears in the content. As one example of the “habit,” use of color, the tendency of drawing of a stroke, the tendency of usage of equipment, the degree of operation error, and so forth are cited. As one example of the “psychological state,” besides emotions including delight, anger, sorrow, and pleasure, various states such as drowsiness, relaxation, and nervousness are cited. As one example of the “external environment,” the ambient brightness, cold and warm, the weather, the season, and so forth are cited.

Further, the content evaluating section 44 obtains the degree of similarity between the time-series feature 52 corresponding to content of an evaluation target (that is, first time-series feature) and the time-series feature 52 corresponding to authentic content (that is, second time-series feature) and determines the authenticity of the content of the evaluation target on the basis of this degree of similarity. For this degree of similarity, for example, various indexes including a correlation coefficient, a norm, and so forth are used.

The output processing section 46 outputs information indicating the evaluation result by the content evaluating section 44 (that is, evaluation result information 54) to the external. In this “output,” besides the case of outputting the information to an output device (not illustrated) disposed in the server device 16 as visible information or audible information, the case of transmitting the information toward an external device such as the user device 12 in FIG. 1 is also included.

The storing section 34 stores the programs and data necessary for the control section 32 to control the respective constituent elements. The storing section 34 is configured by a non-transitory computer-readable storage medium. Here, the computer-readable storage medium is configured from [1] a storage device such as a HDD or SSD incorporated in a computer system, [2] a portable medium such as a magneto-optical disc, ROM, CD-ROM, or flash memory, or the like.

In the example of FIG. 2, in the storing section 34, a database relating to content (hereinafter, content DB 50) is constructed, and multiple sets of the time-series feature 52 and the evaluation result information 54 are stored.

The content data D1 is an aggregate of content elements configuring content and is configured to be capable of expressing the creation process of the content. For example, the content data D1 is formed of ink data (hereinafter, digital ink) for expressing content made by handwriting. As an “ink description language” for describing the digital ink, for example, Wacom Ink Layer Language (WILL), Ink Markup Language (InkML), and Ink Serialized Format (ISF) are cited. The content may be an artwork including a picture, illustrations, characters, and so forth, for example.

The related data D2 includes various pieces of information relating to creation of content. As the related data D2, for example, the following kinds of data are cited: [1] creator information including identification information, attributes, and so forth of the creator of content, [2] “setting conditions of the device driver side” including the resolution, size, and kind of the display unit 24, the detection performance and kind of the touch sensor 25, the shape of a writing pressure curve, and so forth, [3] “setting conditions of the drawing application side” including the kind of content, color information of a color palette and a brush, settings of visual effects, and so forth, [4] “operation history of the creator” sequentially stored through execution of a drawing application, or the like.

The time-series feature 52 indicates time change of a feature relating to the creation process of content and is stored in association with identification information of the content or the creator. Specifically, the time-series feature 52 is an aggregate of data pairs indicating the correspondence relation between at least one kind of feature and the time. The feature is various quantitative values for evaluating the style of content, creator's habit, the psychological state of the creator, the external environment at the time of creation, and so forth. The time may be the actual time including the date and the clock time, the elapsed time from the start timing of creation, or the order of generation or editing of the content elements (for example, order of writing of strokes).

The evaluation result information 54 includes the evaluation result of content by the content evaluating section 44. As one example of the evaluation result, [1] the result of a single-entity evaluation including a classification category, a score, and so forth and [2] the result of a comparative evaluation including the degree of similarity, authenticity determination, and so forth are cited.

### Operation of Content Evaluation System 10

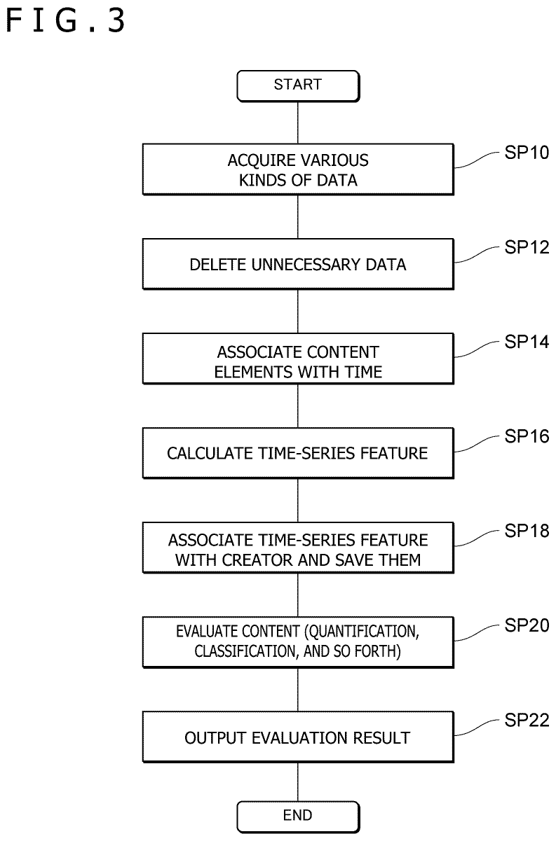

The content evaluation system 10 in this embodiment is configured as above. Subsequently, an operation of the server device 16 configuring part of the content evaluation system 10 will be described with reference to FIGS. 3 to 11.

### First Operation: Calculation of Feature

FIG. 3 is a flowchart illustrating a first operation of the server device 16. This “first operation” means an operation of calculating the feature of content used for an evaluation.

At SP10, the data acquiring section 40 acquires various kinds of data relating to content of an evaluation target, for example, at least one of the content data D1 and the related data D2.

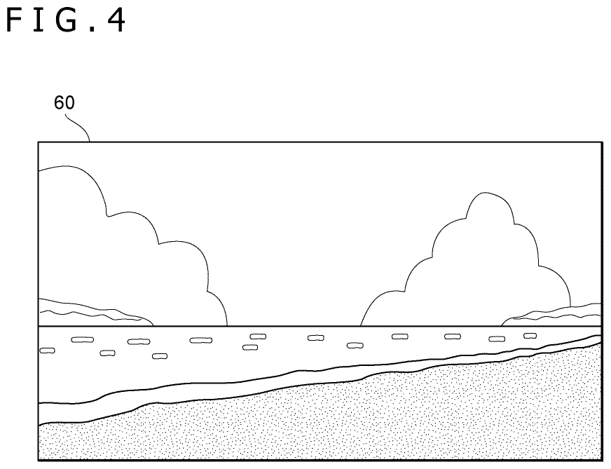

FIG. 4 is a diagram illustrating one example of content made by use of the user device 12 in FIG. 1. In the example of this diagram, the content is an artwork 60 in which a scene of a sandy beach is drawn. The creator completes the desired artwork 60 while using the user device 12 and the electronic pen 14. Even when multiple creators draw a similar scene, the creation process including picture making and use of color differs for each creator.

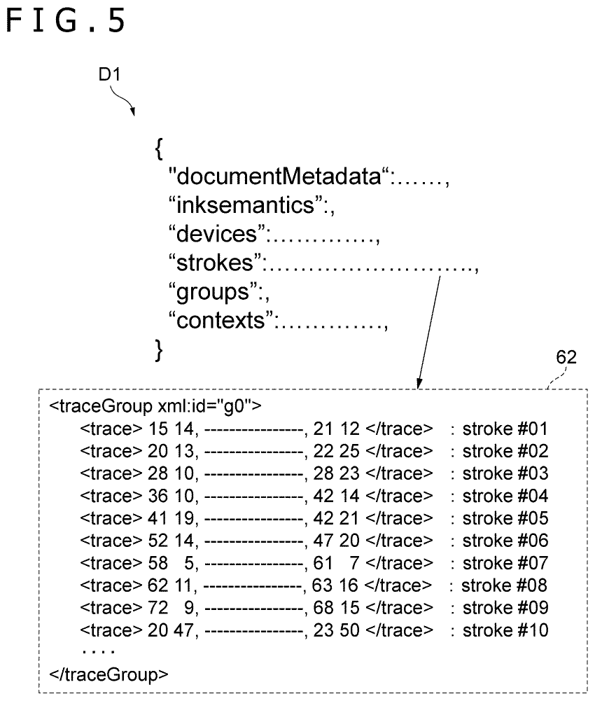

FIG. 5 is a diagram illustrating one example of a data structure that the content data D1 in FIG. 1 has. In the example of this diagram, the case in which the content data D1 is digital ink is illustrated. The digital ink has a data structure obtained by sequentially arranging [1] document metadata (document Metadata), [2] semantic data (ink semantics), [3] device data (devices), [4] stroke data (strokes), [5] classification data (groups), and [6] context data (contexts).

Stroke data 62 is data for describing individual strokes configuring content made by handwriting and indicates the shape of the strokes configuring the content and the order of writing of the strokes. As is understood from FIG. 5, one stroke is described by multiple pieces of point data sequentially arranged in <trace> tags. Each point data is composed of at least an indicated position (X-coordinate, Y-coordinate) and is marked off by a delimiter such as a comma. For convenience of illustration, only the respective pieces of point data indicating the start point and the end point of the stroke are represented, and the pieces of point data indicating multiple passing points are omitted. In this point data, besides the above-described indicated position, the order of generation or editing of the stroke and the writing pressure and the posture of the electronic pen 14, and so forth may be included.

At SP12 in FIG. 3, the feature calculating section 42 executes the removal processing to remove data unnecessary for calculation of the time-series feature 52 to be described later in the various kinds of data acquired at SP10. Specifically, the feature calculating section 42 deletes the content elements deleted in the creation process of the content or data relating to a user operation that does not contribute to the completion of the content. In the “user operation that does not contribute,” for example, a “cancel operation” to cancel an immediately-previous operation error, an “erase operation” to erase a rough, and so forth are included. In the case of evaluating creator's habit, the feature calculating section 42 may leave the above-described user operation as it is without deleting it.

At SP14, the feature calculating section 42 executes the association processing to associate the content elements with the time by using the data shaped by SP12. In the content elements, for example, strokes, figures, text characters, color painting, and so forth are included. For example, when a time stamp is set every user operation, the feature calculating section 42 associates the content element corresponding to the user operation with the actual time identified by the time stamp. Alternatively, when an index indicating the order of writing of each content element is given, the feature calculating section 42 associates the content element with the index.

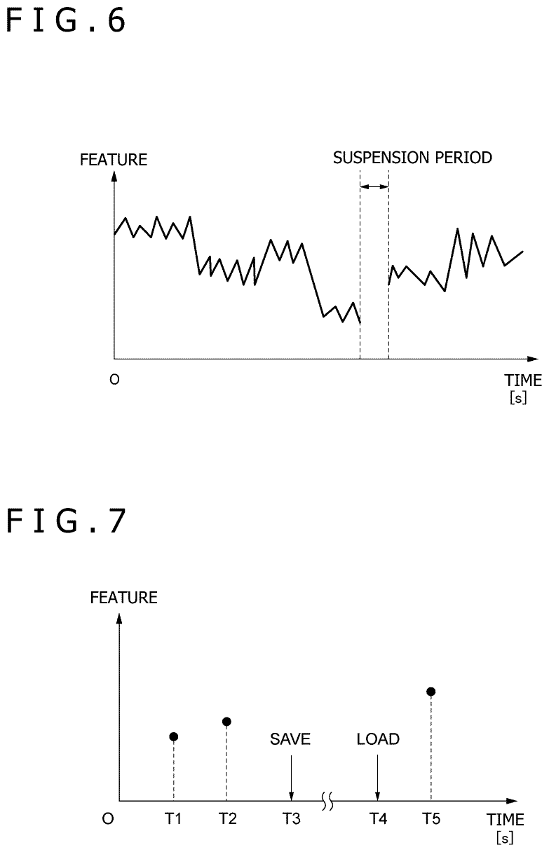

FIG. 6 is a diagram illustrating one example of time change of the feature. The abscissa axis of the graph indicates the time (unit: s), and the ordinate axis of the graph indicates the feature (unit: optional). The feature of this diagram greatly varies in the long term while repeating small variation in the short term. The creator completes content at a stretch in some cases, whereas content is intermittently created with the interposition of a temporary suspension period in other cases.

At SP16 in FIG. 3, the feature calculating section 42 calculates the time-series feature 52 indicating a time change of the feature relating to the creation process of the content by using the dataset associated at SP14. Specifically, the feature calculating section 42 calculates the time-series feature 52 by aggregating one kind or two or more kinds of features for each time. Prior to this calculation, the feature calculating section 42 may execute [1] the “cutting-down processing” to cut down a blank time or [2] the “normalization processing” to execute normalization of the feature and the time, according to need.

FIG. 7 is a diagram illustrating time change of the feature in the case in which a blank time is included in the creation period of content. The definition of the graph is the same as the definition in FIG. 6. One stroke is generated at each of timings T1, and T2, and the content data D1 is saved at a timing T3. After loading of the data is executed at a timing T4, one stroke is generated at a timing T5. Here, if a time difference (T4−T3) is sufficiently larger than a time difference (T2−T1), the whole of the creation time of the content becomes more liable to be affected. As a result, there is a possibility that the evaluation accuracy of the content lowers. Thus, the feature calculating section 42 regards the period from the saving to the loading of the data as a “blank time” included in a time interval of generation or editing of the stroke and cuts down the period.

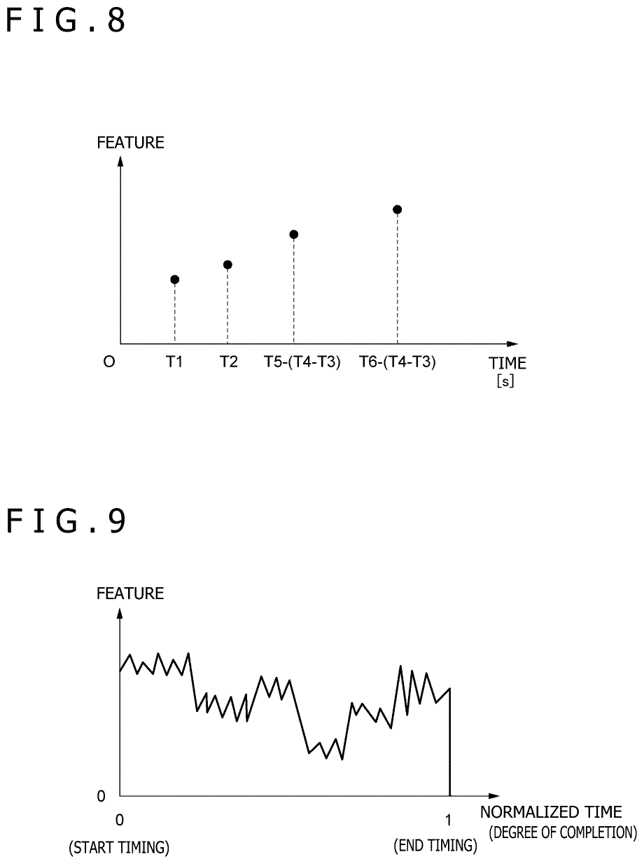

FIG. 8 is a diagram illustrating time change of the feature in the case in which the blank time has been cut down. The timing T5 and the subsequent timings are corrected by cutting down the blank time corresponding to the time difference (T4−T3). Specifically, the original timing T5 is replaced by a timing {T5−(T4−T3)}, and an original timing T6 is replaced by a timing {T6−(T4−T3)}. By cutting down the blank time that is not involved in generation of the content element, the length of the time required for the creation of content can be brought close to a constant length, and correspondingly more accurate capturing of characteristics of the content is facilitated.

FIG. 9 is a diagram illustrating one example of the time-series feature 52 calculated by the feature calculating section 42. The abscissa axis of the graph indicates a normalized time (unit: dimensionless), and the ordinate axis of the graph indicates a normalized feature (unit: dimensionless). Here, the normalized time is a normalized elapsed time from the timing at which creation of content has been started (that is, start timing) and corresponds to the degree of completion of the content. Specifically, the normalized time is defined in such a manner that the value corresponding to the start timing becomes “0” and the value corresponding to the timing at which the creation of the content has ended (that is, end timing) becomes “1.” By setting the length of the time required for the creation of the content (that is, time range) constant in this manner, more accurate capturing of characteristics of the content is facilitated.

Further, when the feature is 8-bit color values (0 to 255) of the CIE RGB color system, the normalized feature is values normalized into a range of [0, 1] by dividing each of the R value, the G value, and the B value by 255, for example. By setting the range of the feature relating to the content elements constant in this manner, more accurate capturing of characteristics of the content is facilitated.

At SP18 in FIG. 3, the feature calculating section 42 supplies the time-series feature 52 calculated at SP16 to the storing section 34 (FIG. 2) together with creator information of the content. This causes the time-series feature 52 to be saved in the content DB 50 in such a state as to be associated with the creator.

At SP20, the content evaluating section 44 executes evaluation processing for evaluating the content by using the time-series feature 52 saved in the content DB 50, according to need. In the flowchart illustrated in FIG. 3, a single-entity evaluation of the content, for example, quantification and classification of the content, is executed.

The stroke data 62 indicating an aggregate of strokes that are the content elements can be included in the content data D1. In this case, the content evaluating section 44 may evaluate at least one of the style of content, the habit of the creator of the content, and the psychological state of the creator by using the time-series feature 52 calculated from at least the stroke data 62 by the feature calculating section 42.

Further, biological data indicating the biological state of the creator at the time of creation of content can be included in the related data D2. In this case, the content evaluating section 44 may evaluate the psychological state of the creator by using the time-series feature 52 calculated from at least the biological data by the feature calculating section 42.

Moreover, environmental data indicating the state of the external environment at the time of creation of content can be included in the related data D2. In this case, the content evaluating section 44 may evaluate the state of the external environment by using the time-series feature 52 calculated from at least the environmental data by the feature calculating section 42.

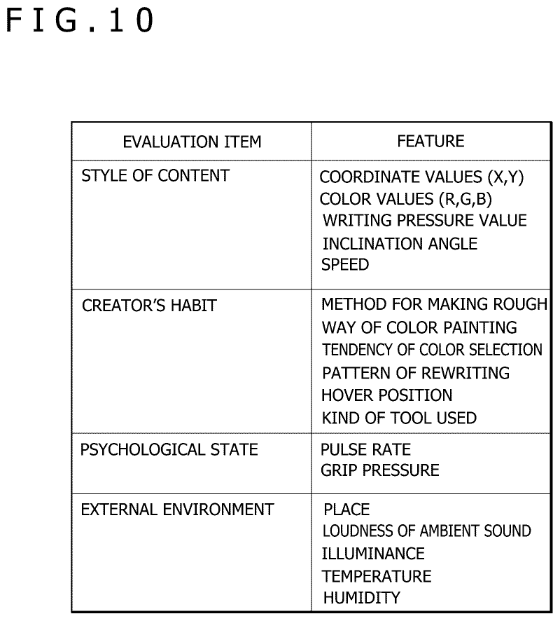

FIG. 10 is a diagram illustrating the correspondence relation between evaluation items of content and the feature suitable for the evaluation items. The kinds of evaluation item and feature and the combinations of these kinds are not limited to the example of this diagram.

As the evaluation items, [1] the style of content, [2] creator's habit, [3] the psychological state of the creator, [4] the external environment at the time of creation, and so forth are cited. As one example of the feature used for the evaluation of the “style,” the coordinate values, the color values, the writing pressure value, the inclination angle, the speed, and so forth are cited. As one example of the feature used for the evaluation of the “habit,” the method for making a rough, the way of color painting, the tendency of color selection, the pattern of rewriting, the hover position of the electronic pen 14, the kind of tool used, and so forth are cited. As one example of the feature used for the evaluation of the “psychological state,” the pulse rate or the heart rate, the grip pressure, and so forth are cited. As one example of the feature used for the evaluation of the “external environment,” the place, the loudness of ambient sound, the illuminance, the temperature, the humidity, and so forth are cited.

At SP22 in FIG. 3, the output processing section 46 outputs the evaluation result obtained at SP20. Specifically, the output processing section 46 transmits data including the evaluation result information 54 toward an external device (for example, user device 12). This allows the creator to visually recognize the evaluation result of the created content through the display unit 24 (FIG. 1) of the creator's user device 12.

In the above-described manner, the server device 16 ends the first operation illustrated in the flowchart of FIG. 3. The evaluation result information 54 is saved in the content DB 50 in such a state as to be associated with the content of the evaluation target and the time-series feature 52.

### Second Operation: Authenticity Determination of Content

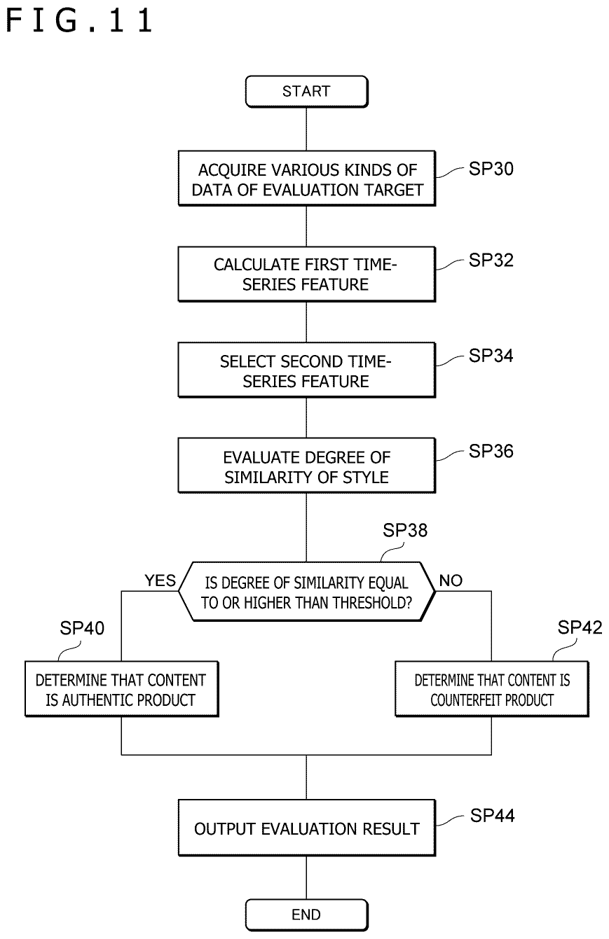

FIG. 11 is a flowchart illustrating a second operation by the server device 16. This “second operation” means an operation relating to authenticity determination to determine whether or not content of an evaluation target is the authentic product.

At SP30, the data acquiring section 40 acquires various kinds of data (here, at least one of the content data D1 and the related data D2) relating to the content of the evaluation target, as in the case of step SP10 in FIG. 3.

At S32, the feature calculating section 42 calculates the time-series feature 52 of the evaluation target (that is, first time-series feature) by using the various kinds of data acquired in step SP30. This calculation processing is executed as in the case of SP12 to SP16 in FIG. 3.

At SP34, the content evaluating section 44 acquires the time-series feature 52 as a comparison target of the first time-series feature calculated at SP32 (that is, second time-series feature). Specifically, the content evaluating section 44 reads out the time-series feature 52 associated with identification information of authentic content or identification information of the creator (that is, second time-series feature) from the content DB 50 to acquire the time-series feature 52.

At SP36, the content evaluating section 44 computes the degree of similarity between the first time-series feature calculated at SP32 and the second time-series feature acquired at SP34. Here, the degree of similarity is defined in such a manner that the style of the content is closer when the value of the degree of similarity is larger, whereas the style of the content is more different when the value is smaller.

At SP38, the content evaluating section 44 checks whether or not the degree of similarity obtained at SP36 is equal to or higher than a threshold. When the degree of similarity is equal to or higher than the threshold (SP38: YES), the content evaluating section 44 determines that the content of the evaluation target is the authentic product (S40), and proceeds to SP44. On the other hand, when the degree of similarity is lower than the threshold (SP38: NO), the content evaluating section 44 determines that the content of the evaluation target is a counterfeit product (S42), and proceeds to the next step SP44.

At S44, the output processing section 46 outputs the determination result obtained by SP40 or SP42. Specifically, the output processing section 46 outputs data including the evaluation result information 54 to the output device (not illustrated) as visible information or audible information. This allows the provider of the content evaluation service to recognize whether or not the content of the evaluation target is the authentic product.

### Summarization of Embodiment

As above, the content evaluation system 10 in this embodiment includes one or multiple user devices 12 capable of generating the content data D1 indicating content (for example, artwork 60) composed of multiple content elements and the content evaluation device (here, server device 16) configured to be capable of communicating with each user device 12.

Further, the server device 16 includes the data acquiring section 40 that acquires at least one of the content data D1 indicating content and the related data D2 relating to creation of the content, the feature calculating section 42 that calculates the time-series feature 52 indicating a time change of the feature relating to the creation process of the content from the acquired content data D1 or related data D2, and the content evaluating section 44 that evaluates the content by using the calculated time-series feature 52.

Moreover, according to a content evaluation computer-readable medium or method in this embodiment, one or multiple computers (or processors) execute an acquisition (SP10) of acquiring at least one of the content data D1 and the related data D2, a calculation (SP16) of calculating the time-series feature 52 from the acquired content data D1 or related data D2, and an evaluation (SP20 or SP36) of evaluating content by use of the calculated time-series feature 52.

By evaluating content by use of the time-series feature 52 indicating a time change of the feature relating to the creation process of the content in this manner, the content can be evaluated more elaborately, compared with the case of executing evaluation merely by use of the contents of drawing of a finished product.

Further, when the content data D1 includes the stroke data 62 indicating an aggregate of strokes that are the content elements, the content evaluating section 44 may evaluate at least one of the style of content, the habit of the creator of the content, and the psychological state of the creator by using the time-series feature 52 calculated from at least the stroke data 62 by the feature calculating section 42. By capturing time-series characteristics relating to the aggregate of the strokes, the style, the habit, or the psychological state can be evaluated more elaborately.

Moreover, when the related data D2 includes biological data indicating the biological state of a creator at the time of creation of content, the content evaluating section 44 may evaluate the psychological state of the creator by using the time-series feature 52 calculated from at least the biological data by the feature calculating section 42. By capturing time-series characteristics relating to the biological state of the creator, the psychological state can be evaluated more elaborately.

Furthermore, when the related data D2 includes environmental data indicating the state of the external environment at the time of creation of content, the content evaluating section 44 may evaluate the state of the external environment by using the time-series feature 52 calculated from at least the environmental data by the feature calculating section 42. By capturing characteristics relating to the state of the external environment, the psychological state can be evaluated more elaborately.

Moreover, the content evaluating section 44 may obtain the degree of similarity between the first time-series feature corresponding to content of an evaluation target and the second time-series feature corresponding to the authentic content and evaluate the authenticity of the content of the evaluation target on the basis of the degree of similarity. By executing the authenticity determination of the content by use of the time-series feature 52, more elaborate determination can be executed, compared with the case of executing determination merely by use of the contents of drawing of a finished product.

Further, the feature calculating section 42 may normalize the time corresponding to each feature in the range from the start timing to the end timing of creation of content and calculate the time-series feature 52. By setting the length of the time required for the creation of the content (that is, time range) constant, more accurate capturing of characteristics of the content is facilitated.

Moreover, the feature calculating section 42 may cut down a blank time included in a time interval of generation or editing of the content element and calculate the time-series feature 52. By cutting down the blank time that is not involved in generation or editing of the content element, the length of the time required for the creation of content can be brought close to a constant length, and correspondingly more accurate capturing of characteristics of the content is facilitated.

### Modification Examples

It is obvious that the present disclosure is not limited to the above-described embodiment and can freely be changed without departing from the gist of this disclosure. Alternatively, the respective configurations may be optionally combined in a range in which no contradiction is caused technically. Alternatively, the order of execution of the respective acts configuring the flowchart may be changed in a range in which no contradiction is caused technically.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A content evaluation device comprising:
a processor; and
a memory storing a program that, when executed by the processor, causes the content evaluation device to:
acquire at least one of content data indicating content composed of multiple content elements or related data relating to creation of the content;
calculate a time-series feature indicating a time change of a feature relating to a creation process of the content from the content data or the related data; and
evaluate the content by using the time-series feature.

2. The content evaluation device according to claim 1, wherein:
the content data includes stroke data indicating an aggregate of strokes that are the content elements, and
the program, when executed by the processor, causes the content evaluation device to evaluate at least one of a style of the content, a habit of a creator of the content, or a psychological state of the creator by using the time-series feature calculated from at least the stroke data.

3. The content evaluation device according to claim 1, wherein:
the related data includes biological data indicating a biological state of a creator at a time of the creation of the content, and
the program, when executed by the processor, causes the content evaluation device to evaluate a psychological state of the creator by using the time-series feature calculated from at least the biological data.

4. The content evaluation device according to claim 1, wherein:
the related data includes environmental data indicating a state of an external environment at a time of the creation of the content, and
the program, when executed by the processor, causes the content evaluation device to evaluate the state of the external environment by using the time-series feature calculated from at least the environmental data.

5. The content evaluation device according to claim 1, wherein:
the program, when executed by the processor, causes the content evaluation device to obtain a degree of similarity between a first time-series feature corresponding to content of an evaluation target and a second time-series feature corresponding to authentic content and evaluates authenticity of the content of the evaluation target based on the degree of similarity.

6. The content evaluation device according to claim 1, wherein:
the program, when executed by the processor, causes the content evaluation device to normalize a plurality of times corresponding to each of the time-series features in a range from a start timing to an end timing of the creation of the content and calculates the time-series feature.

7. The content evaluation device according to claim 1, wherein:
the program, when executed by the processor, causes the content evaluation device to cut down a blank time included in a time interval of generation or editing of the content elements and calculates the time-series feature.

8. A non-transitory computer-readable medium storing a content evaluation program that, when executed by one or more computers, causes the one or more computers to:
acquire at least one of content data indicating content composed of multiple content elements or related data relating to creation of the content;
calculate a time-series feature indicating a time change of a feature relating to a creation process of the content from the content data or the related data; and
evaluate the content by using the time-series feature.

9. A content evaluation method comprising:
acquiring, by one or more computers, at least one of content data indicating content composed of multiple content elements or related data relating to creation of the content;
calculating, by the one or more computers, a time-series feature indicating a time change of a feature relating to a creation process of the content from the content data or the related data; and
evaluating, by the one or more computers, the content by use of the time-series feature.

10. A content evaluation system comprising:
a user device that, in operation, generates content data indicating content composed of multiple content elements; and
a server device that, in operation, communicates with the user device,
wherein the server device includes:
a processor; and
a memory storing a program that, when executed by the processor, causes the server device to:
acquire at least one of the content data or related data relating to creation of the content from the user device,
calculate a time-series feature indicating a time change of a feature relating to a creation process of the content from the content data or the related data, and
evaluate the content by using the time-series feature.
